(19) United States
(12) Patent Application Publication
(10) Pub. No.: US 2023/0398169 A1
(43) Pub. Date: Dec. 14, 2023

(54) NOVEL PROCESS

(71) Applicant: SpecNova LLC, Evergreen, CO (US)

(72) Inventor: Sebastian BALCOMBE, Evergreen, CO (US)

(73) Assignee: SpecNova LLC, Evergreen, CO (US)

(21) Appl. No.: 18/329,272

(22) Filed: Jun. 5, 2023

Related U.S. Application Data
(60) Provisional application No. 63/366,086, filed on Jun. 9, 2022.

(51) Int. Cl. A61K 36/736; A61K 47/24; A61K 47/36; A61K 31/222; A61K 31/7048; A61K 31/192

(52) U.S. Cl. CPC A61K 36/736; A61K 47/24; A61K 47/36; A61K 31/222; A61K 31/7048; A61K 31/192; A61K 2236/333; A61K 2236/37

(57) ABSTRACT

This invention relates to a process for preparing a tart cherry extract composition by combining a tart cherry extract with a rosmarinic acid extract. Related compositions and methods are also provided.

### CROSS REFERENCE TO RELATED APPLICATIONS

This application claims priority to U.S. provisional application No. 63/366,086, filed on Jun. 9, 2022, the entire contents of which are hereby incorporated by reference herein.

### FIELD OF THE INVENTION

The invention relates to a process for preparing a tart cherry extract composition. The invention also relates to a tart cherry extract composition obtainable by the aforementioned process, nutraceutical and pharmaceutical compositions comprising said extract composition, and uses of said nutraceutical and pharmaceutical compositions.

### BACKGROUND OF THE INVENTION

Tart cherries (Prunus cerasus L.), also known as sour, dwarf or Montmorency cherries, are a species of Prunus widely distributed throughout Europe and southwest Asia. It is smaller than the sweet cherry tree, has twiggy branches, and bears crimson-to-near-black cherries. There are two main varieties of tart cherries: the dark-red Morello cherry and the yellow flesh coloured Amarelle cherry. The fruit is closely related to sweet cherries (Prunus avium), but its fruit tends to be more acidic with an edible pulp inside with a pleasant aroma. As opposed to sweet cherries, which tend to be enjoyed fresh, tart cherries are often eaten dried, frozen, or juiced.

Tart cherries contain many biologically active chemical constituents including polyphenols such as anthocyanins. However, tart cherry anthocyanins are unstable and have limited oral bioavailability.

There is therefore a need to provide alternative tart cherry extraction processes which result in extracts with improved properties.

### SUMMARY OF THE INVENTION

According to the first aspect of the invention, there is provided a process for preparing a tart cherry extract composition, which comprises combining a tart cherry extract with a rosmarinic acid extract.

According to a further aspect of the invention there is provided a tart cherry extract composition obtainable by the processes described herein.

According to a further aspect of the invention there is provided a nutraceutical composition comprising the extract composition as described herein, and one or more nutraceutically acceptable excipients.

According to a further aspect of the invention there is provided a pharmaceutical composition comprising the extract composition as described herein, and one or more pharmaceutically acceptable excipients.

### BRIEF DESCRIPTION OF THE FIGURES

FIG. 1: A representative example of the process for preparing a tart cherry extract composition claimed herein.

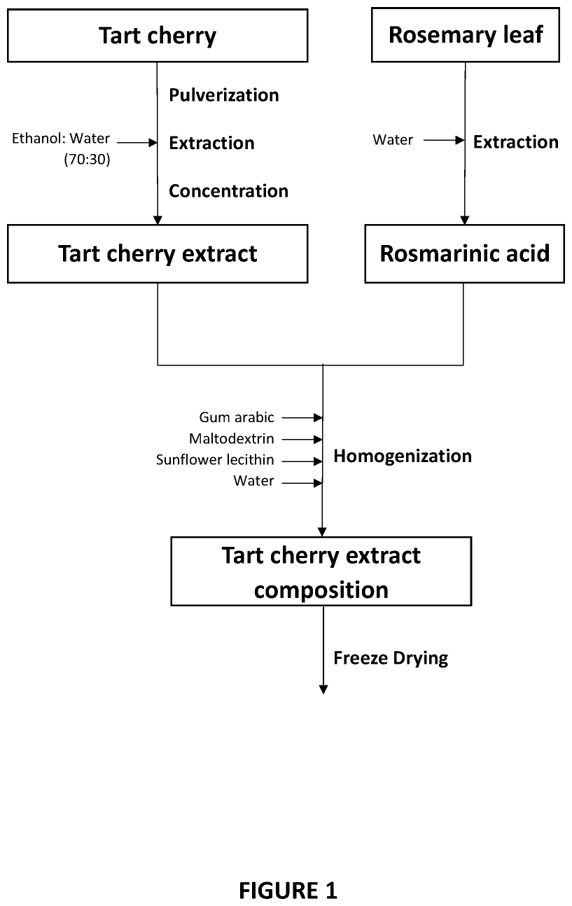

### DETAILED DESCRIPTION OF THE INVENTION

Process

According to the first aspect of the invention, there is provided process for preparing a tart cherry extract composition, which comprises combining a tart cherry extract with a rosmarinic acid extract. This process has a number of advantages. By complexing the anthocyanins extracted from tart cherry with rosmarinic acid from rosemary, the anthocyanins are protected from degradation, enhancing the bioavailability and thereby efficacy of the anthocyanins.

In one embodiment, the process described herein comprises the steps of (a) extracting pulverised tart cherries with an aqueous alcohol solution followed by evaporation of the extract solution to obtain a concentrated tart cherry extract; (b) subjecting rosemary to hot water extraction followed by filtration and acidification of the extract solution to obtain a rosmarinic acid precipitate; (c) combining the product of step (a) with the product of step (b), a stabiliser, a thickening agent, an emulsifier, and water, followed by subjecting the mixture to homogenisation.

In one embodiment, the process further comprises spray drying the product of step (c).

In one embodiment, the aqueous alcohol solution is an ethyl alcohol:water mixture. In further embodiments the ethyl alcohol:water mixture is a 70:30 ethyl alcohol:water mixture.

In one embodiment, the stabiliser is gum arabic.

In one embodiment, the thickening agent is maltodextrin.

In one embodiment, the emulsifier is lecithin. In further embodiments, the lecithin is sunflower lecithin.

In one embodiment, the final pH value of the acidification step of step (c) is 2.0.

Composition

According to a further aspect of the invention there is provided a tart cherry extract composition obtainable by the processes described herein.

In one embodiment, the tart cherry extract composition comprises at least one of cyanidin-3-glucosylrutinoside, cyanidin-3-rutinoside, cyanidin sophoroside, chlorogenic acid, neochlorogenic acid, 3′-p-coumaroylquinic acid, 3-caffeoylquinic acid, 5-caffeoylquinic acid, p-coumaric acid, kaempferol-3-rutinoside, quercetin-3-glucoside, quercetin-3-rutinoside, quercetin-3-(2-glucosyl-rutinoside) and isorhamnetin rutinoside, or a salt or solvate of any one thereof.

References herein to “cyanidin-3-glucosylrutinoside” refer to a compound having the following structure:

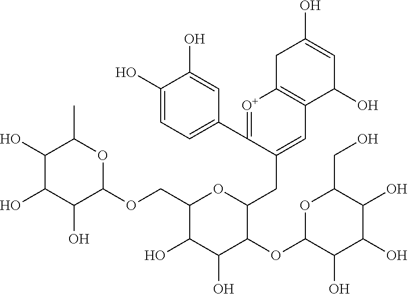 [SMILES: CC1OC(OCC2OC(Cc3cc4c([o+]c3-c3ccc(O)c(O)c3)CC(O)=CC4O)C(OC3OC(CO)C(O)C(O)C3O)C(O)C2O)C(O)C(O)C1O]

References herein to “cyanidin-3-rutinoside” refer to a compound having the following structure:

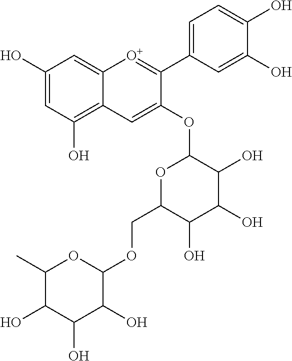 [SMILES: CC1OC(OCC2OC(Oc3cc4c(O)cc(O)cc4[o+]c3-c3ccc(O)c(O)c3)C(O)C(O)C2O)C(O)C(O)C1O]

References herein to “cyanidin sophoroside” refer to a compound having the following structure:

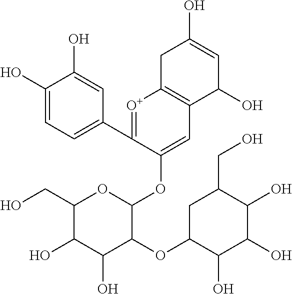 [SMILES: OCC1CC(OC2C(Oc3cc4c([o+]c3-c3ccc(O)c(O)c3)CC(O)=CC4O)OC(CO)C(O)C2O)C(O)C(O)C1O]

References herein to “chlorogenic acid” refer to a compound having the following structure:

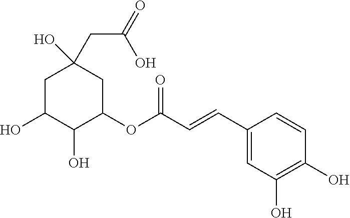 [SMILES: O=C(O)CC1(O)CC(O)C(O)C(OC(=O)/C=C/c2ccc(O)c(O)c2)C1]

References herein to “neochlorogenic acid” refer to a compound having the following structure:

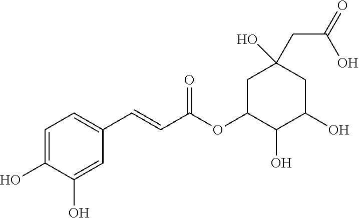 [SMILES: O=C(O)CC1(O)CC(O)C(O)C(OC(=O)/C=C/c2ccc(O)c(O)c2)C1]

References herein to “3′-p-coumaroylquinic acid” refer to a compound having the following structure:

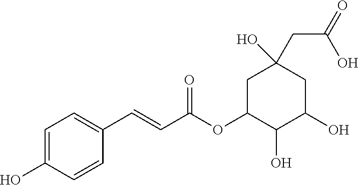 [SMILES: O=C(O)CC1(O)CC(O)C(O)C(OC(=O)/C=C/c2ccc(O)cc2)C1]

References herein to “3-caffeoylquinic acid” refer to a compound having the following structure:

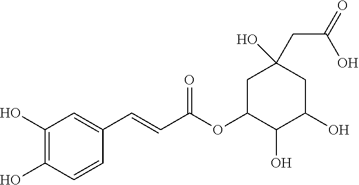 [SMILES: O=C(O)CC1(O)CC(O)C(O)C(OC(=O)/C=C/c2ccc(O)c(O)c2)C1]

References herein to “5-caffeoylquinic acid” refer to a compound having the following structure:

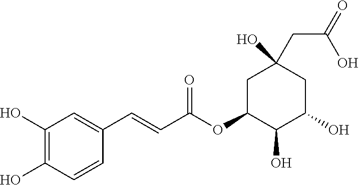 [SMILES: O=C(O)C[C@@]1(O)C[C@H](OC(=O)/C=C/c2ccc(O)c(O)c2)[C@H](O)[C@@H](O)C1]

References herein to “p-coumaric acid” refer to a compound having the following structure:

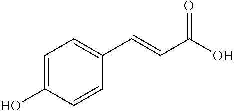 [SMILES: O=C(O)/C=C/c1ccc(O)cc1]

References herein to “kaempferol-3-rutinoside” refer to a compound having the following structure:

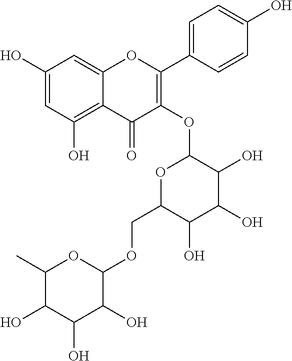 [SMILES: CC1OC(OCC2OC(Oc3c(-c4ccc(O)cc4)oc4cc(O)cc(O)c4c3=O)C(O)C(O)C2O)C(O)C(O)C1O]

References herein to “quercetin-3-glucoside” refer to a compound having the following structure:

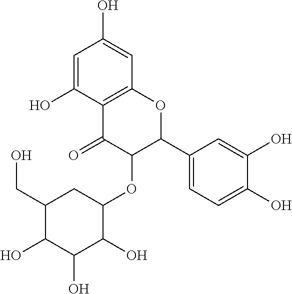 [SMILES: O=C1c2c(O)cc(O)cc2OC(c2ccc(O)c(O)c2)C1OC1CC(CO)C(O)C(O)C1O]

References herein to “quercetin-3-rutinoside” refer to a compound having the following structure:

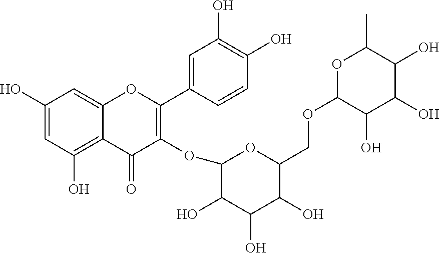 [SMILES: CC1OC(OCC2OC(Oc3c(-c4ccc(O)c(O)c4)oc4cc(O)cc(O)c4c3=O)C(O)C(O)C2O)C(O)C(O)C1O]

References herein to “quercetin-3-(2-glucosyl-rutinoside)” refer to a compound having the following structure:

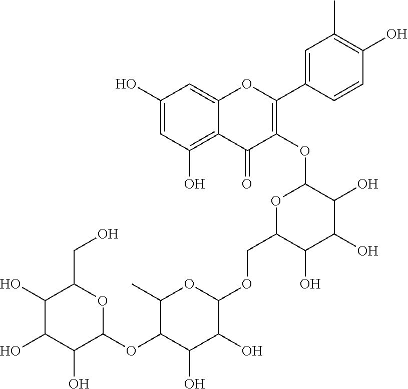 [SMILES: Cc1cc(-c2oc3cc(O)cc(O)c3c(=O)c2OC2OC(COC3OC(C)C(OC4OC(CO)C(O)C(O)C4O)C(O)C3O)C(O)C(O)C2O)ccc1O]

References herein to “isorhamnetin rutinoside” refer to a compound having the following structure:

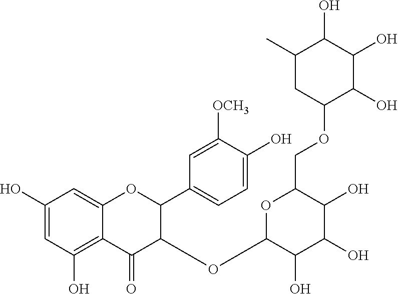 [SMILES: COc1cc(C2Oc3cc(O)cc(O)c3C(=O)C2OC2OC(COC3CC(C)C(O)C(O)C3O)C(O)C(O)C2O)ccc1O]

In one embodiment, the tart cherry extract composition comprises at least two of cyanidin-3-glucosylrutinoside, cyanidin-3-rutinoside, cyanidin sophoroside, chlorogenic acid, neochlorogenic acid, 3′-p-coumaroylquinic acid, 3-caffeoylquinic acid, 5-caffeoylquinic acid, p-coumaric acid, kaempferol-3-rutinoside, quercetin-3-glucoside, quercetin-3-rutinoside, quercetin-3-(2-glucosyl-rutinoside) and isorhamnetin rutinoside, or a salt or solvate of any one thereof.

In one embodiment, the tart cherry extract composition comprises at least three of cyanidin-3-glucosylrutinoside, cyanidin-3-rutinoside, cyanidin sophoroside, chlorogenic acid, neochlorogenic acid, 3′-p-coumaroylquinic acid, 3-caffeoylquinic acid, 5-caffeoylquinic acid, p-coumaric acid, kaempferol-3-rutinoside, quercetin-3-glucoside, quercetin-3-rutinoside, quercetin-3-(2-glucosyl-rutinoside) and isorhamnetin rutinoside, or a salt or solvate of any one thereof.

In one embodiment, the tart cherry extract composition comprises at least four of cyanidin-3-glucosylrutinoside, cyanidin-3-rutinoside, cyanidin sophoroside, chlorogenic acid, neochlorogenic acid, 3′-p-coumaroylquinic acid, 3-caffeoylquinic acid, 5-caffeoylquinic acid, p-coumaric acid, kaempferol-3-rutinoside, quercetin-3-glucoside, quercetin-3-rutinoside, quercetin-3-(2-glucosyl-rutinoside) and isorhamnetin rutinoside, or a salt or solvate of any one thereof.

In one embodiment, the tart cherry extract composition comprises at least five of cyanidin-3-glucosylrutinoside, cyanidin-3-rutinoside, cyanidin sophoroside, chlorogenic acid, neochlorogenic acid, 3′-p-coumaroylquinic acid, 3-caffeoylquinic acid, 5-caffeoylquinic acid, p-coumaric acid, kaempferol-3-rutinoside, quercetin-3-glucoside, quercetin-3-rutinoside, quercetin-3-(2-glucosyl-rutinoside) and isorhamnetin rutinoside, or a salt or solvate of any one thereof.

In one embodiment, the tart cherry extract composition comprises at least six of cyanidin-3-glucosylrutinoside, cyanidin-3-rutinoside, cyanidin sophoroside, chlorogenic acid, neochlorogenic acid, 3′-p-coumaroylquinic acid, 3-caffeoylquinic acid, 5-caffeoylquinic acid, p-coumaric acid, kaempferol-3-rutinoside, quercetin-3-glucoside, quercetin-3-rutinoside, quercetin-3-(2-glucosyl-rutinoside) and isorhamnetin rutinoside, or a salt or solvate of any one thereof.

In one embodiment, the tart cherry extract composition comprises at least seven of cyanidin-3-glucosylrutinoside, cyanidin-3-rutinoside, cyanidin sophoroside, chlorogenic acid, neochlorogenic acid, 3′-p-coumaroylquinic acid, 3-caffeoylquinic acid, 5-caffeoylquinic acid, p-coumaric acid, kaempferol-3-rutinoside, quercetin-3-glucoside, quercetin-3-rutinoside, quercetin-3-(2-glucosyl-rutinoside) and isorhamnetin rutinoside, or a salt or solvate of any one thereof.

In one embodiment, the tart cherry extract composition comprises at least eight of cyanidin-3-glucosylrutinoside, cyanidin-3-rutinoside, cyanidin sophoroside, chlorogenic acid, neochlorogenic acid, 3′-p-coumaroylquinic acid, 3-caffeoylquinic acid, 5-caffeoylquinic acid, p-coumaric acid, kaempferol-3-rutinoside, quercetin-3-glucoside, quercetin-3-rutinoside, quercetin-3-(2-glucosyl-rutinoside) and isorhamnetin rutinoside, or a salt or solvate of any one thereof.

In one embodiment, the tart cherry extract composition comprises at least nine of cyanidin-3-glucosylrutinoside, cyanidin-3-rutinoside, cyanidin sophoroside, chlorogenic acid, neochlorogenic acid, 3′-p-coumaroylquinic acid, 3-caffeoylquinic acid, 5-caffeoylquinic acid, p-coumaric acid, kaempferol-3-rutinoside, quercetin-3-glucoside, quercetin-3-rutinoside, quercetin-3-(2-glucosyl-rutinoside) and isorhamnetin rutinoside, or a salt or solvate of any one thereof.

In one embodiment, the tart cherry extract composition comprises at least ten of cyanidin-3-glucosylrutinoside, cyanidin-3-rutinoside, cyanidin sophoroside, chlorogenic acid, neochlorogenic acid, 3′-p-coumaroylquinic acid, 3-caffeoylquinic acid, 5-caffeoylquinic acid, p-coumaric acid, kaempferol-3-rutinoside, quercetin-3-glucoside, quercetin-3-rutinoside, quercetin-3-(2-glucosyl-rutinoside) and isorhamnetin rutinoside, or a salt or solvate of any one thereof.

In one embodiment, the tart cherry extract composition comprises at least eleven of cyanidin-3-glucosylrutinoside, cyanidin-3-rutinoside, cyanidin sophoroside, chlorogenic acid, neochlorogenic acid, 3′-p-coumaroylquinic acid, 3-caffeoylquinic acid, 5-caffeoylquinic acid, p-coumaric acid, kaempferol-3-rutinoside, quercetin-3-glucoside, quercetin-3-rutinoside, quercetin-3-(2-glucosyl-rutinoside) and isorhamnetin rutinoside, or a salt or solvate of any one thereof.

In one embodiment, the tart cherry extract composition comprises at least twelve of cyanidin-3-glucosylrutinoside, cyanidin-3-rutinoside, cyanidin sophoroside, chlorogenic acid, neochlorogenic acid, 3′-p-coumaroylquinic acid, 3-caffeoylquinic acid, 5-caffeoylquinic acid, p-coumaric acid, kaempferol-3-rutinoside, quercetin-3-glucoside, quercetin-3-rutinoside, quercetin-3-(2-glucosyl-rutinoside) and isorhamnetin rutinoside, or a salt or solvate of any one thereof.

In one embodiment, the tart cherry extract composition comprises at least thirteen of cyanidin-3-glucosylrutinoside, cyanidin-3-rutinoside, cyanidin sophoroside, chlorogenic acid, neochlorogenic acid, 3′-p-coumaroylquinic acid, 3-caffeoylquinic acid, 5-caffeoylquinic acid, p-coumaric acid, kaempferol-3-rutinoside, quercetin-3-glucoside, quercetin-3-rutinoside, quercetin-3-(2-glucosyl-rutinoside) and isorhamnetin rutinoside, or a salt or solvate of any one thereof.

In one embodiment, the tart cherry extract composition comprises each of cyanidin-3-glucosylrutinoside, cyanidin-3-rutinoside, cyanidin sophoroside, chlorogenic acid, neochlorogenic acid, 3′-p-coumaroylquinic acid, 3-caffeoylquinic acid, 5-caffeoylquinic acid, p-coumaric acid, kaempferol-3-rutinoside, quercetin-3-glucoside, quercetin-3-rutinoside, quercetin-3-(2-glucosyl-rutinoside) and isorhamnetin rutinoside, or a salt or solvate of any one thereof.

Nutraceutical Compositions

According to a further aspect of the invention there is provided a nutraceutical composition comprising the extract composition as described herein and one or more nutraceutically acceptable excipients.

References herein to a nutraceutical refer to a food, food product, food additive or dietary supplement that provides health and/or medical benefits, such as preventing, treating and enhancing mammalian (e.g. human) conditions. References herein to food extend equally to a drink or beverage comprising said nutraceutical.

In one embodiment, the nutraceutical composition additionally comprises one or more additional active ingredients.

In one embodiment, the nutraceutical composition is a tablet or capsule.

In one embodiment, the nutraceutical composition is a food or beverage selected from: water, milk, coffee, tea, juice, protein shake, energy drink, yoghurt and cereal or chocolate bar.

In one embodiment, the nutraceutical composition is for use as a food, food product, food additive or dietary supplement.

The nutraceutically acceptable excipient(s) can be selected from, for example, carriers (e.g. a solid, liquid or semi-solid carrier), adjuvants, diluents, fillers or bulking agents, granulating agents, coating agents, release-controlling agents, binding agents, disintegrants, lubricating agents, preservatives, antioxidants, buffering agents, suspending agents, thickening agents, flavouring agents, sweeteners, taste masking agents, stabilisers or any other excipients conventionally used in pharmaceutical compositions. Examples of excipients for various types of nutraceutical compositions are set out in more detail below.

The term “nutraceutically acceptable” as used herein pertains to compounds, materials, compositions, and/or dosage forms which are, within the scope of sound medical judgment, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Nutraceutical compositions containing compounds of the invention can be formulated in accordance with known techniques, see for example, Remington's Pharmaceutical Sciences, Mack Publishing Company, Easton, PA, USA.

The nutraceutical compositions can be administered to the subject in need thereof in any suitable and convenient form. Suitably, said administration will be orally or topically.

Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, vegetable oils (such as sunflower oil, safflower oil, corn oil or olive oil), and injectable organic esters such as ethyl oleate. Proper fluidity can be maintained, for example, by the use of thickening or coating materials such as lecithin, by the maintenance of the required particle size in the case of dispersions, and by the use of surfactants.

The compositions of the present invention may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol, sorbic acid, and the like. It may also be desirable to include agents to adjust tonicity such as sugars, sodium chloride, and the like.

Nutraceutical dosage forms suitable for oral administration include tablets (coated or uncoated), capsules (hard or soft shell), caplets, pills, lozenges, syrups, solutions, powders, granules, elixirs and suspensions, sublingual tablets, wafers or patches such as buccal patches.

Thus, tablet compositions can contain a unit dosage of active compound together with an inert diluent or carrier such as a sugar or sugar alcohol, eg; lactose, sucrose, sorbitol or mannitol; and/or a non-sugar derived diluent such as sodium carbonate, calcium phosphate, calcium carbonate, or a cellulose or derivative thereof such as microcrystalline cellulose (MCC), methyl cellulose, ethyl cellulose, hydroxypropyl methyl cellulose, and starches such as corn starch. Tablets may also contain such standard ingredients as binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures.

Tablets may be designed to release the active compound either upon contact with stomach fluids (immediate release tablets) or to release in a controlled manner (controlled release tablets) over a prolonged period of time or with a specific region of the GI tract.

Capsule formulations may be of the hard gelatin or soft gelatin variety and can contain the active component in solid, semi-solid, or liquid form. Gelatin capsules can be formed from animal gelatin or synthetic or plant derived equivalents thereof.

The solid dosage forms (eg; tablets, capsules etc.) can be coated or un-coated. Coatings may act either as a protective film (e.g. a polymer, wax or varnish) or as a mechanism for controlling drug release or for aesthetic or identification purposes. The coating (e.g. a Eudragit™ type polymer) can be designed to release the active component at a desired location within the gastro-intestinal tract. Thus, the coating can be selected so as to degrade under certain pH conditions within the gastrointestinal tract, thereby selectively release the compound in the stomach or in the ileum, duodenum, jejenum or colon.

Instead of, or in addition to, a coating, the active compound can be presented in a solid matrix comprising a release controlling agent, for example a release delaying agent which may be adapted to release the compound in a controlled manner in the gastrointestinal tract. Alternatively the drug can be presented in a polymer coating e.g. a polymethacrylate polymer coating, which may be adapted to selectively release the compound under conditions of varying acidity or alkalinity in the gastrointestinal tract. Alternatively, the matrix material or release retarding coating can take the form of an erodible polymer (e.g. a maleic anhydride polymer) which is substantially continuously eroded as the dosage form passes through the gastrointestinal tract. In another alternative, the coating can be designed to disintegrate under microbial action in the gut. As a further alternative, the active compound can be formulated in a delivery system that provides osmotic control of the release of the compound. Osmotic release and other delayed release or sustained release formulations (for example formulations based on ion exchange resins) may be prepared in accordance with methods well known to those skilled in the art.

The compounds of the invention may be formulated with a carrier and administered in the form of nanoparticles, the increased surface area of the nanoparticles assisting their absorption. In addition, nanoparticles offer the possibility of direct penetration into the cell. Nanoparticle drug delivery systems are described in “Nanoparticle Technology for Drug Delivery”, edited by Ram B Gupta and Uday B. Kompella, Informa Healthcare, ISBN 9781574448573, published 13 Mar. 2006. Nanoparticles for drug delivery are also described in J. Control. Release, 2003, 91 (1-2), 167-172, and in Sinha et al., Mol. Cancer Ther. August 1, (2006) 5, 1909.

The nutraceutical compositions typically comprise from approximately 1% (w/w) to approximately 95% (w/w) active ingredient and from 99% (w/w) to 5% (w/w) of a nutraceutically acceptable excipient or combination of excipients. Particularly, the compositions comprise from approximately 20% (w/w) to approximately 90%,% (w/w) active ingredient and from 80% (w/w) to 10% of a nutraceutically acceptable excipient or combination of excipients. The nutraceutical compositions comprise from approximately 1% to approximately 95%, particularly from approximately 20% to approximately 90%, active ingredient.

The nutraceutically acceptable excipient(s) can be selected according to the desired physical form of the formulation and can, for example, be selected from diluents (e.g solid diluents such as fillers or bulking agents; and liquid diluents such as solvents and co-solvents), disintegrants, buffering agents, lubricants, flow aids, release controlling (e.g. release retarding or delaying polymers or waxes) agents, binders, granulating agents, pigments, plasticizers, antioxidants, preservatives, flavouring agents, taste masking agents, tonicity adjusting agents and coating agents.

The skilled person will have the expertise to select the appropriate amounts of ingredients for use in the formulations. For example tablets and capsules typically contain 0-20% disintegrants, 0-5% lubricants, 0-5% flow aids and/or 0-99% (w/w) fillers/or bulking agents (depending on drug dose). They may also contain 0-10% (w/w) polymer binders, 0-5% (w/w) antioxidants, 0-5% (w/w) pigments. Slow release tablets would in addition contain 0-99% (w/w) release-controlling (e.g. delaying) polymers (depending on dose). The film coats of the tablet or capsule typically contain 0-10% (w/w) polymers, 0-3% (w/w) pigments, and/or 0-2% (w/w) plasticizers.

Nutraceutical compositions for oral administration can be obtained by combining the active ingredient with solid carriers, if desired granulating a resulting mixture, and processing the mixture, if desired or necessary, after the addition of appropriate excipients, into tablets, dragee cores or capsules. It is also possible for them to be incorporated into a polymer or waxy matrix that allow the active ingredients to diffuse or be released in measured amounts.

The compounds of the invention can also be formulated as solid dispersions. Solid dispersions are homogeneous extremely fine disperse phases of two or more solids. Solid solutions (molecularly disperse systems), one type of solid dispersion, are well known for use in pharmaceutical technology (see (Chiou and Riegelman, J. Pharm. Sci., 60, 1281-1300 (1971)) and are useful in increasing dissolution rates and increasing the bioavailability of poorly water-soluble drugs.

This invention also provides solid dosage forms comprising the solid solution described above. Solid dosage forms include tablets, capsules, chewable tablets and dispersible or effervescent tablets. Known excipients can be blended with the solid solution to provide the desired dosage form. For example, a capsule can contain the solid solution blended with (a) a disintegrant and a lubricant, or (b) a disintegrant, a lubricant and a surfactant. In addition a capsule can contain a bulking agent, such as lactose or microcrystalline cellulose. A tablet can contain the solid solution blended with at least one disintegrant, a lubricant, a surfactant, a bulking agent and a glidant. A chewable tablet can contain the solid solution blended with a bulking agent, a lubricant, and if desired an additional sweetening agent (such as an artificial sweetener), and suitable flavours. Solid solutions may also be formed by spraying solutions of drug and a suitable polymer onto the surface of inert carriers such as sugar beads (‘non-pareils’). These beads can subsequently be filled into capsules or compressed into tablets.

Compositions for topical use and nasal delivery include ointments, creams, sprays, patches, gels, liquid drops and inserts (for example intraocular inserts). Such compositions can be formulated in accordance with known methods.

The compounds of the invention will generally be presented in unit dosage form and, as such, will typically contain sufficient compound to provide a desired level of activity. For example, a formulation may contain from 1 nanogram to 2 grams of active ingredient, e.g. from 1 nanogram to 2 milligrams of active ingredient. Within these ranges, particular sub-ranges of compound are 0.1 milligrams to 2 grams of active ingredient (more usually from 10 milligrams to 1 gram, e.g. 50 milligrams to 500 milligrams), or 1 microgram to 20 milligrams (for example 1 microgram to 10 milligrams, e.g. 0.1 milligrams to 2 milligrams of active ingredient).

For oral compositions, a unit dosage form may contain from 1 milligram to 2 grams, more typically 10 milligrams to 1 gram, for example 50 milligrams to 1 gram, e.g. 100 miligrams to 1 gram, of active compound.

The active compound will be administered to a patient in need thereof (for example a human or animal patient) in an amount sufficient to achieve the desired effect.

Although it is anticipated that the nutraceutical composition of the invention will be present within a tablet or capsule, it may also be within a food or beverage. Examples of suitable foods or beverages where the nutraceutical compositions may be contained within include: water, milk, coffee, tea, juice, protein shake, energy drink, yoghurt, cereal or chocolate bar, and the like.

Nutraceutical Utility

Tart cherry has many antioxidant and anti-inflammatory polyphenol compounds. Tart cherry has been demonstrated to lessen pain and accelerate strength recovery after exercise and decrease blood markers of inflammation/oxidative stress.

Therefore, according to a further aspect of the invention there is provided the nutraceutical composition as described herein for use in improving or increasing one or more of the following: exercise performance, joint mobility, sleep quality, and sleep duration; or reduction of one or more of the following: free radicals, oxidative stress, inflammation, creatine kinase activity, creatine kinase myocardial band levels, muscle damage and muscle soreness.

Pharmaceutical Compositions

According to a further aspect of the invention there is provided a pharmaceutical composition comprising the extract composition as described herein, and one or more pharmaceutically acceptable excipients.

In one embodiment, the pharmaceutical composition additionally comprises one or more additional active ingredients.

The pharmaceutically acceptable excipient(s) can be selected from, for example, carriers (e.g. a solid, liquid or semi-solid carrier), adjuvants, diluents, fillers or bulking agents, granulating agents, coating agents, release-controlling agents, binding agents, disintegrants, lubricating agents, preservatives, antioxidants, buffering agents, suspending agents, thickening agents, flavouring agents, sweeteners, taste masking agents, stabilisers or any other excipients conventionally used in pharmaceutical compositions. Examples of excipients for various types of pharmaceutical compositions are set out in more detail below.

The term “pharmaceutically acceptable” as used herein pertains to compounds, materials, compositions, and/or dosage forms which are, within the scope of sound medical judgment, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Pharmaceutical compositions containing compounds of the invention can be formulated in accordance with known techniques, see for example, Remington's Pharmaceutical Sciences, Mack Publishing Company, Easton, PA, USA.

The pharmaceutical compositions can be in any form suitable for oral, parenteral, topical, intranasal, intrabronchial, sublingual, ophthalmic, otic, rectal, intra-vaginal, or transdermal administration. Where the compositions are intended for parenteral administration, they can be formulated for intravenous, intramuscular, intraperitoneal, subcutaneous administration or for direct delivery into a target organ or tissue by injection, infusion or other means of delivery. The delivery can be by bolus injection, short term infusion or longer-term infusion and can be via passive delivery or through the utilisation of a suitable infusion pump or syringe driver.

Pharmaceutical formulations adapted for parenteral administration include aqueous and non-aqueous sterile injection solutions which may contain anti-oxidants, buffers, bacteriostats, co-solvents, surface active agents, organic solvent mixtures, cyclodextrin complexation agents, emulsifying agents (for forming and stabilizing emulsion formulations), liposome components for forming liposomes, gellable polymers for forming polymeric gels, lyophilisation protectants and combinations of agents for, inter alia, stabilising the active ingredient in a soluble form and rendering the formulation isotonic with the blood of the intended recipient. Pharmaceutical formulations for parenteral administration may also take the form of aqueous and non-aqueous sterile suspensions which may include suspending agents and thickening agents (R. G. Strickly, Solubilizing Excipients in oral and injectable formulations, Pharmaceutical Research, Vol 21(2) 2004, p 201-230).

The formulations may be presented in unit-dose or multi-dose containers, for example sealed ampoules, vials and prefilled syringes, and may be stored in a freeze-dried (lyophilised) condition requiring only the addition of the sterile liquid carrier, for example water for injections, immediately prior to use. In one embodiment, the formulation is provided as an active pharmaceutical ingredient in a bottle for subsequent reconstitution using an appropriate diluent.

The pharmaceutical formulation can be prepared by lyophilising a compound of the invention. Lyophilisation refers to the procedure of freeze-drying a composition. Freeze-drying and lyophilisation are therefore used herein as synonyms.

Extemporaneous injection solutions and suspensions may be prepared from sterile powders, granules and tablets.

Pharmaceutical compositions of the present invention for parenteral injection can also comprise pharmaceutically acceptable sterile aqueous or non-aqueous solutions, dispersions, suspensions or emulsions as well as sterile powders for reconstitution into sterile injectable solutions or dispersions just prior to use.

Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, vegetable oils (such as sunflower oil, safflower oil, corn oil or olive oil), and injectable organic esters such as ethyl oleate. Proper fluidity can be maintained, for example, by the use of thickening or coating materials such as lecithin, by the maintenance of the required particle size in the case of dispersions, and by the use of surfactants.

The compositions of the present invention may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol, sorbic acid, and the like. It may also be desirable to include agents to adjust tonicity such as sugars, sodium chloride, and the like. Prolonged absorption of the injectable pharmaceutical form may be brought about by the inclusion of agents which delay absorption such as aluminum monostearate and gelatin.

Pharmaceutical dosage forms suitable for oral administration include tablets (coated or uncoated), capsules (hard or soft shell), caplets, pills, lozenges, syrups, solutions, powders, granules, elixirs and suspensions, sublingual tablets, wafers or patches such as buccal patches.

Thus, tablet compositions can contain a unit dosage of active compound together with an inert diluent or carrier such as a sugar or sugar alcohol, eg; lactose, sucrose, sorbitol or mannitol; and/or a non-sugar derived diluent such as sodium carbonate, calcium phosphate, calcium carbonate, or a cellulose or derivative thereof such as microcrystalline cellulose (MCC), methyl cellulose, ethyl cellulose, hydroxypropyl methyl cellulose, and starches such as corn starch. Tablets may also contain such standard ingredients as binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures.

Tablets may be designed to release the drug either upon contact with stomach fluids (immediate release tablets) or to release in a controlled manner (controlled release tablets) over a prolonged period of time or with a specific region of the GI tract. Capsule formulations may be of the hard gelatin or soft gelatin variety and can contain the active component in solid, semi-solid, or liquid form. Gelatin capsules can be formed from animal gelatin or synthetic or plant derived equivalents thereof.

The solid dosage forms (eg; tablets, capsules etc.) can be coated or un-coated. Coatings may act either as a protective film (e.g. a polymer, wax or varnish) or as a mechanism for controlling drug release or for aesthetic or identification purposes. The coating (e.g. a Eudragit™ type polymer) can be designed to release the active component at a desired location within the gastro-intestinal tract. Thus, the coating can be selected so as to degrade under certain pH conditions within the gastrointestinal tract, thereby selectively release the compound in the stomach or in the ileum, duodenum, jejenum or colon.

Instead of, or in addition to, a coating, the drug can be presented in a solid matrix comprising a release controlling agent, for example a release delaying agent which may be adapted to release the compound in a controlled manner in the gastrointestinal tract. Alternatively the drug can be presented in a polymer coating e.g. a polymethacrylate polymer coating, which may be adapted to selectively release the compound under conditions of varying acidity or alkalinity in the gastrointestinal tract. Alternatively, the matrix material or release retarding coating can take the form of an erodible polymer (e.g. a maleic anhydride polymer) which is substantially continuously eroded as the dosage form passes through the gastrointestinal tract. In another alternative, the coating can be designed to disintegrate under microbial action in the gut. As a further alternative, the active compound can be formulated in a delivery system that provides osmotic control of the release of the compound. Osmotic release and other delayed release or sustained release formulations (for example formulations based on ion exchange resins) may be prepared in accordance with methods well known to those skilled in the art.

The compounds of the invention may be formulated with a carrier and administered in the form of nanoparticles, the increased surface area of the nanoparticles assisting their absorption. In addition, nanoparticles offer the possibility of direct penetration into the cell. Nanoparticle drug delivery systems are described in “Nanoparticle Technology for Drug Delivery”, edited by Ram B Gupta and Uday B. Kompella, Informa Healthcare, ISBN 9781574448573, published 13 Mar. 2006. Nanoparticles for drug delivery are also described in J. Control. Release, 2003, 91 (1-2), 167-172, and in Sinha et al., Mol. Cancer Ther. August 1, (2006) 5, 1909.

The pharmaceutical compositions typically comprise from approximately 1% (w/w) to approximately 95% (w/w) active ingredient and from 99% (w/w) to 5% (w/w) of a pharmaceutically acceptable excipient or combination of excipients. Particularly, the compositions comprise from approximately 20% (w/w) to approximately 90%,% (w/w) active ingredient and from 80% (w/w) to 10% of a pharmaceutically acceptable excipient or combination of excipients. The pharmaceutical compositions comprise from approximately 1% to approximately 95%, particularly from approximately 20% to approximately 90%, active ingredient. Pharmaceutical compositions according to the invention may be, for example, in unit dose form, such as in the form of ampoules, vials, suppositories, pre-filled syringes, dragees, tablets or capsules.

The pharmaceutically acceptable excipient(s) can be selected according to the desired physical form of the formulation and can, for example, be selected from diluents (e.g solid diluents such as fillers or bulking agents; and liquid diluents such as solvents and co-solvents), disintegrants, buffering agents, lubricants, flow aids, release controlling (e.g. release retarding or delaying polymers or waxes) agents, binders, granulating agents, pigments, plasticizers, antioxidants, preservatives, flavouring agents, taste masking agents, tonicity adjusting agents and coating agents.

The skilled person will have the expertise to select the appropriate amounts of ingredients for use in the formulations. For example tablets and capsules typically contain 0-20% disintegrants, 0-5% lubricants, 0-5% flow aids and/or 0-99% (w/w) fillers/or bulking agents (depending on drug dose). They may also contain 0-10% (w/w) polymer binders, 0-5% (w/w) antioxidants, 0-5% (w/w) pigments. Slow release tablets would in addition contain 0-99% (w/w) release-controlling (e.g. delaying) polymers (depending on dose). The film coats of the tablet or capsule typically contain 0-10% (w/w) polymers, 0-3% (w/w) pigments, and/or 0-2% (w/w) plasticizers.

Parenteral formulations typically contain 0-20% (w/w) buffers, 0-50% (w/w) cosolvents, and/or 0-99% (w/w) Water for Injection (WFI) (depending on dose and if freeze dried). Formulations for intramuscular depots may also contain 0-99% (w/w) oils.

Pharmaceutical compositions for oral administration can be obtained by combining the active ingredient with solid carriers, if desired granulating a resulting mixture, and processing the mixture, if desired or necessary, after the addition of appropriate excipients, into tablets, dragee cores or capsules. It is also possible for them to be incorporated into a polymer or waxy matrix that allow the active ingredients to diffuse or be released in measured amounts.

The compounds of the invention can also be formulated as solid dispersions. Solid dispersions are homogeneous extremely fine disperse phases of two or more solids. Solid solutions (molecularly disperse systems), one type of solid dispersion, are well known for use in pharmaceutical technology (see (Chiou and Riegelman, J. Pharm. Sci., 60, 1281-1300 (1971)) and are useful in increasing dissolution rates and increasing the bioavailability of poorly water-soluble drugs.

This invention also provides solid dosage forms comprising the solid solution described above. Solid dosage forms include tablets, capsules, chewable tablets and dispersible or effervescent tablets. Known excipients can be blended with the solid solution to provide the desired dosage form. For example, a capsule can contain the solid solution blended with (a) a disintegrant and a lubricant, or (b) a disintegrant, a lubricant and a surfactant. In addition a capsule can contain a bulking agent, such as lactose or microcrystalline cellulose. A tablet can contain the solid solution blended with at least one disintegrant, a lubricant, a surfactant, a bulking agent and a glidant. A chewable tablet can contain the solid solution blended with a bulking agent, a lubricant, and if desired an additional sweetening agent (such as an artificial sweetener), and suitable flavours. Solid solutions may also be formed by spraying solutions of drug and a suitable polymer onto the surface of inert carriers such as sugar beads (‘non-pareils’). These beads can subsequently be filled into capsules or compressed into tablets.

The pharmaceutical formulations may be presented to a patient in “patient packs” containing an entire course of treatment in a single package, usually a blister pack. Patient packs have an advantage over traditional prescriptions, where a pharmacist divides a patient's supply of a pharmaceutical from a bulk supply, in that the patient always has access to the package insert contained in the patient pack, normally missing in patient prescriptions. The inclusion of a package insert has been shown to improve patient compliance with the physician's instructions.

Compositions for topical use and nasal delivery include ointments, creams, sprays, patches, gels, liquid drops and inserts (for example intraocular inserts). Such compositions can be formulated in accordance with known methods.

Examples of formulations for rectal or intra-vaginal administration include pessaries and suppositories which may be, for example, formed from a shaped moldable or waxy material containing the active compound. Solutions of the active compound may also be used for rectal administration.

Compositions for administration by inhalation may take the form of inhalable powder compositions or liquid or powder sprays, and can be administrated in standard form using powder inhaler devices or aerosol dispensing devices. Such devices are well known. For administration by inhalation, the powdered formulations typically comprise the active compound together with an inert solid powdered diluent such as lactose.

The compounds of the invention will generally be presented in unit dosage form and, as such, will typically contain sufficient compound to provide a desired level of biological activity. For example, a formulation may contain from 1 nanogram to 2 grams of active ingredient, e.g. from 1 nanogram to 2 milligrams of active ingredient. Within these ranges, particular sub-ranges of compound are 0.1 milligrams to 2 grams of active ingredient (more usually from 10 milligrams to 1 gram, e.g. 50 milligrams to 500 milligrams), or 1 microgram to 20 milligrams (for example 1 microgram to 10 milligrams, e.g. 0.1 milligrams to 2 milligrams of active ingredient).

For oral compositions, a unit dosage form may contain from 1 milligram to 2 grams, more typically 10 milligrams to 1 gram, for example 50 milligrams to 1 gram, e.g. 100 miligrams to 1 gram, of active compound.

The active compound will be administered to a patient in need thereof (for example a human or animal patient) in an amount sufficient to achieve the desired therapeutic effect.

Therapeutic Utility

According to a further aspect of the invention, there is provided the pharmaceutical composition as defined herein, for use in therapy.

According to a further aspect of the invention there is provided the pharmaceutical composition as defined herein, for use in the prophylaxis or treatment of one or more of the following: arthritis, gout.

The invention will now be described with reference to the following non-limiting examples:

### Example 1: Preparation of Tart Cherries

10 kg of tart cherries were extracted with 50 L of aqueous ethanol solution (70:30 ethanol:water) for 3 hours. The solvent was subsequently evaporated to yield approximately 1.2 kg tart cherry extract.

### Example 2—Preparation of Rosemary

Rosemary leaves were defatted using a conventional solid-liquid extraction performed with 95% hexane at a ratio of sample to solvent of 1:5 (w/v) in an Ultraturrax (11,000 rpm) for 5 min. The mixture was then centrifuged at 4500 rpm for 10 min at 20° C. The supernatant was removed, and the precipitate was extracted again following the same procedure. The final precipitate sample was completely dried to remove any residual hexane by flushing with nitrogen gas for 5 min and heating at 30° C. for 10 min in an orbital incubator

400 g of the defatted, dried and powdered rosemary leaves were subjected to water extraction with 2 L of water at 80° C. for an hour with stirring. The extract was then filtered and acidified to pH 2 with 25% HCl. The resulting precipitate was collected and dried in a vacuum oven, yielding 30 g of rosmarinic acid.

### Example 3—Preparation of the Combined Extract

1.2 kg of tart cherry extract prepared in Example 1 was combined with 30 g of the rosmarinic acid extract prepared in Example 2, along with 200 g of gum arabic and 400 g of maltodextrin. 3 L of water was added to the mixture, resulting in mix 1. Separately, 60 g of sunflower lecithin was dissolved in 2 L water with a high shear mixer, resulting in mix 2. Mix 1 and mix 2 were then homogenized and complexed.

1. A process for preparing a tart cherry extract composition, which comprises combining a tart cherry extract with a rosmarinic acid extract.

2. The process of claim 1, which comprises the steps of:
(a) extracting pulverised tart cherries with an aqueous alcohol solution followed by evaporation of the extract solution to obtain a concentrated tart cherry extract;
(b) subjecting rosemary to hot water extraction followed by filtration and acidification of the extract solution to obtain a rosmarinic acid precipitate;
(c) combining the product of step (a) with the product of step (b), a stabiliser, a thickening agent, an emulsifier, and water, followed by subjecting the mixture to homogenisation.

3. The process of claim 2, which further comprises spray drying the product of step (c).

4. The process of claim 2, wherein the aqueous alcohol solution is an ethyl alcohol:water mixture.

5. The process of claim 2, wherein the stabiliser is gum arabic.

6. The process of claim 2, wherein the thickening agent is maltodextrin.

7. The process of claim 2, wherein the emulsifier is lecithin.

8. The process of claim 7, where the lecithin is sunflower lecithin.

9. A tart cherry extract composition prepared by the process of claim 1.

10. The tart cherry extract composition of claim 9, which comprises at least one of cyanidin-3-glucosylrutinoside, cyanidin-3-rutinoside, cyanidin sophoroside, chlorogenic acid, neochlorogenic acid, 3′-p-coumaroylquinic acid, 3-caffeoylquinic acid, 5-caffeoylquinic acid, p-coumaric acid, kaempferol-3-rutinoside, quercetin-3-glucoside, quercetin-3-rutinoside, quercetin-3-(2-glucosyl-rutinoside) and isorhamnetin rutinoside, or a salt or solvate of any one thereof.

11. A nutraceutical composition comprising the tart cherry extract composition according to claim 9 and one or more nutraceutically acceptable excipients.

12. The nutraceutical composition according to claim 11, which additionally comprises one or more additional active ingredients.

13. The nutraceutical composition according to claim 11, which is a tablet or capsule.

14. The nutraceutical composition according to claim 11, which is a food or beverage selected from: water, milk, coffee, tea, juice, protein shake, energy drink, yoghurt and cereal or chocolate bar.

15. A food, food product, food additive or dietary supplement comprising the nutraceutical composition according to claim 11.

16. A method for improving or increasing one or more of the following: exercise performance, joint mobility, sleep quality, and sleep duration; or reduction of one or more of the following: free radicals, oxidative stress, inflammation, creatine kinase activity, creatine kinase myocardial band levels, or muscle damage and muscle soreness, said method comprising administering the nutraceutical composition according to claim 11.

17. A pharmaceutical composition comprising the tart cherry extract composition according to claim 9 and one or more pharmaceutically acceptable excipients.

18. The pharmaceutical composition according to claim 17, in combination with one or more therapeutic agents.

19. A method for treating arthritis or gout comprising administering the pharmaceutical composition according to claim 17.

20. The process of claim 2, wherein:
the aqueous alcohol solution is an ethyl alcohol:water mixture;
the stabiliser is gum arabic;
the thickening agent is maltodextrin; and
the emulsifier is lecithin.
